(19) United States
(12) Patent Application Publication
(10) Pub. No.: US 2026/0076921 A1
(43) Pub. Date: Mar. 19, 2026

(54) Transdermal Pharmaceutical Formulations for the Treatment of Multiple Sclerosis

(71) Applicant: Pike Therapeutics Inc., Vancouver (CA)

(72) Inventors: Fotios M. PLAKOGIANNIS, Whitestone, NY (US); Tamanna LATHER, Jersey City, NJ (US)

(21) Appl. No.: 19/399,983

(22) Filed: Nov. 25, 2025

Related U.S. Application Data
(62) Division of application No. 17/365,060, filed on Jul. 1, 2021.
(60) Provisional application No. 63/046,855, filed on Jul. 1, 2020.

(51) Int. Cl. A61K 9/70; A61K 9/00; A61K 31/00; A61K 31/137; A61K 31/277; A61P 25/28

(52) U.S. Cl. CPC A61K 9/7015; A61K 9/0021; A61K 9/7053; A61K 9/7061; A61K 9/7069; A61K 9/7084; A61K 31/137; A61K 31/277; A61K 31/658; A61P 25/28

(57) ABSTRACT

The present disclosure relates to the to the transdermal administration of THC and/or CBD and derivatives of these compounds, for the treatment and/or prevention and/or control of multiple sclerosis, multiple sclerosis-related muscle spasms, and pain and/or spasticity in multiple sclerosis.

This application is a Divisional of U.S. Ser. No. 17/365,060 filed Jul. 1, 2021, which claims priority to U.S. Ser. No. 63/046,855 filed Jul. 1, 2020, the entirety of which is incorporated herein by reference.

### BACKGROUND OF THE INVENTION

Multiple sclerosis (MS) is an autoimmune disease that affects the central nervous system (CNS). The CNS consists of the brain, spinal cord, and the optic nerves. Surrounding and protecting the nerve fibers of the CNS is a fatty tissue called myelin that helps nerve fibers conduct electrical impulses. In MS, myelin is lost in multiple areas, leaving scar tissue called sclerosis. These damaged areas are also known as plaques or lesions. In some cases, the nerve fiber itself is damaged or broken. When myelin or the nerve fiber is destroyed or damaged, the ability of the nerves to conduct electrical impulses to and from the brain is disrupted, and this produces the various symptoms of MS. Patients with MS can expect one of four clinical courses of disease: relapsing-remitting, primary-progressive, secondary-progressive or progressive-relapsing.

Neurodegenerative diseases, such as MS, are a group of disorders characterized by changes in normal neuronal functioning, leading, in most cases, to neuronal death. Most of these diseases are associated, especially in late stages, with severe neuronal loss. With an ever-increasing ageing population, progressively more individuals are affected by neurodegenerative diseases. According to the National Institute of Neurological Disorders and Stroke, there are more than 600 different types of neurological disorders.

Neural degeneration, or neurodegeneration, can be described as the progressive damage or death of neurons. Neurons are nerve cells in the brain whose primary function is to assist in the memory process. The damage or death of neurons leads to a gradual deterioration of the functions controlled by the affected part of the nervous system. Neural degeneration often occurs because of oxidative stress. Oxidative stress occurs to the cells when the effects of pro-oxidants (such as free radicals, reactive oxygen and reactive nitrogen species) exceed the ability of anti-oxidants to neutralize them. When levels of free radicals or other pro-oxidants increase to such an extent, they can cause damage to cell membranes which in turn may result in cell death or damage to genetic material.

Some of the most common types of neurological disorders include Alzheimer's disease, Parkinson's disease and MS. The process of neural degeneration is often the result of glutamate excitotoxicity. Glutamate is a signaling chemical and under normal conditions the concentration of glutamate in a cell tends to be quite low. Glutamate is required at these low concentrations for crucial brain functions such as memory and learning. When glutamate concentrations increase, the process of neural degeneration begins.

Several pharmaceutical products exist which contain either phytocannabinoids (natural) or synthetic cannabinoids. For example, dronabinol (Marinol) is the International Nonproprietary Name (INN) for an encapsulated THC product which has been used therapeutically as an appetite stimulant, antiemetic, and analgesic, as an oral drug. Also, nabilone (Cesamet) is a synthetic analog of dronabinol (Marinol), while Sativex is a cannabinoid extract oral spray containing THC, and other cannabinoids that are used to treat neuropathic pain and spasticity. Sativex (combination of THC and cannabidiol) is approved in some countries for the improvement of spasticity symptoms due to multiple sclerosis. Sativex an oromucosal spray is administered multiple times a day that is ranging from one spray/day to 12 sprays/day¹. Further, rimonabant (marketed under various tradenames) is a selective cannabinoid receptor antagonist used as an anti-obesity drug and as a smoking cessation. Several other cannabinoid-containing products exist.

Thus, considering the therapeutic effect of compounds containing cannabinoids, especially (—)-Δ⁹-trans-THC, there is a continuing need for improving existing cannabinoid-containing products as well as a need for new products containing cannabinoids, especially in the pharmaceutical field.

Clearly, due to multiple oral dosing per day there remains a pressing and long felt need in the art of developing treatment options for multiple sclerosis, multiple sclerosis-related muscle spasms, and pain and/or spasticity in multiple sclerosis in a patient, in particular, for the development of a novel and effective pharmaceutical composition for use in the treatment of chronic pain, the composition inflicting less side effects to the patient.

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannabinol (CBN), cannabichromene (CBC), cannabigerol (CBG), tetrahydrocannbivarin (THCV), delta 9-tetrahydrocannabinol (Δ⁹THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation.

There is a need for an improved drug delivery system of CBD and/or THC for treatment of spasticity due to multiple sclerosis which can overcome the drawbacks associated with oral routes that is multiple dosing per day. Transdermal delivery of CBD and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, solution thereof in solvents alone or in combinations thereof can address the challenges associated with oral drug delivery, and are useful as treatment of for example, multiple sclerosis, multiple sclerosis-related muscle spasms, and pain and/or spasticity in multiple sclerosis in a patient.

All references cited herein are incorporated herein by reference in their entireties.

### BRIEF SUMMARY OF THE INVENTION

The disclosure provides compositions and methods for the treatment and/or prevention and/or control of multiple sclerosis, multiple sclerosis-related muscle spasms, and pain and/or spasticity in multiple sclerosis, using transdermal drug delivery. In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week.

Through transdermal delivery, transdermal compositions or transdermal formulations or transdermal patch of, for example, of THC and/or CBD can be applied topically to skin thereby delivering the drug throughout the duration of topical application. Depending on the requirement, the duration of topical application can be once in a day, once in two days, once in three days, once in four days, once in five days, once in a week. Therefore, transdermal delivery can overcome the multiple dose regimen of oral delivery by reducing the dosing frequency.

Moreover, in transdermal drug delivery the drug is delivered slowly and continuously throughout the duration of topical application hence there are no peaks and troughs in drug plasma concentration which are associated with multiple dose administration in a day. Therefore, by transdermal delivery of CBD and/or THC, patients can have the therapeutic effect of the drug for extended period of time without drastic changes in drug plasma concentration.

In transdermal delivery drug is delivered into systemic circulation through the skin, it escapes the first pass hepatic metabolism therefore to achieve the desired therapeutic activity less drug is required, resulting into less adverse effects or side effects.

Furthermore, transdermal delivery is easy, noninvasive and convenient. Administration of a transdermal patch or transdermal composition does not require medical supervision as patients can topically apply the transdermal patch or transdermal composition themselves. Therefore, transdermal delivery can overcome the drawbacks of injections which are often painful and requires medical supervision.

With respect to THC and/or CBD it is expected that interpatient variability in pharmacologic response will be less with transdermal delivery as drug plasma concentration can be controlled by controlling the rate of drug delivery from transdermal composition or transdermal patch. With transdermal delivery a small amount of THC and/or CBD can be delivered for longer duration than oral administration. Transdermal formulations of CBD and/or THC also provide more abuse deterrence than immediate release dosage forms.

Moreover, in case of any adverse effect, side effect or emergency transdermal delivery gives the liberty to terminate the therapy anytime by taking off the transdermal patch or transdermal composition from skin.

As per above stated reasons for the treatment and/or prevention and/or control of of multiple sclerosis, multiple sclerosis-related muscle spasms, and pain and/or spasticity in multiple sclerosis, transdermal delivery can provide patient friendly, simplified and convenient therapeutic regimen over traditional delivery systems. Transdermal delivery can reduce the dosing frequency of active agent as defined herein. Depending on the necessity, dosing frequency can be once in a day, once in two days, once in three days, once in four days, once in five days, once in six days, once in a week.

Through transdermal administration of drug combination, two or more drugs can be delivered simultaneously. By combining drugs which are administered for treating relapsing form of multiple sclerosis can provide a much-simplified dosage regimen to patients. Combination of drugs can be such as combination of THC, CBD, teriflunomide, combination of THC, CBD, fingolimod or its salt. Depending on the necessity, dosing frequency of transdermal patch or transdermal composition containing drug combination can be once in a day, once in two days, once in three days, once in four days, once in five days, once in six days, once in a week. It would be a great addition to the patient compliance.

The disclosure provides a pharmaceutical composition comprising active agent tetrahydrocannabinol (THC), cannabidiol (CBD) alone or in combinations thereof in a dosage form for transdermal delivery. The disclosure provides a pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, stereoisomers thereof, solid solution thereof, solution thereof, powder form thereof, liquid form thereof, alone or combinations thereof. The disclosure provides a pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, stereoisomers thereof, solid solution thereof, solution thereof, powder form thereof, liquid form thereof, alone or combinations thereof. The disclosure provides a pharmaceutical composition which comprises at least about 0.5% to about 70% (w/w) of the active agent. The disclosure provides a pharmaceutical composition which comprises at least about 2% to about 30% of the active agent. The disclosure provides a pharmaceutical composition which comprises at least about 90% to about 99% (w/w) of the active agent. The disclosure provides a pharmaceutical composition which comprises active agent at a concentration selected from the group consisting of about 90%, about 91%, about 92%, about 93%, about 94%, about 95%, about 96%, about 97%, about 98%, and about 99% (w/w). The disclosure provides a pharmaceutical composition formulated as transdermal liquid formulation, transdermal semisolid formulation, transdermal gel formulation, or transdermal matrix formulation. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, surfactants, antioxidants, oxidants, and combinations thereof. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, surfactants, antioxidants, oxidants, and combinations thereof in the range of 0.5%-98% w/w or w/v. The disclosure provides a pharmaceutical composition wherein the carrier is present in the range of 70%-98% w/w or w/v. The disclosure provides a pharmaceutical composition which is formulated as a transdermal patch. The disclosure provides a pharmaceutical composition which is formulated as a metered dose transdermal gel, metered dose transdermal spray. The disclosure provides a pharmaceutical composition formulated as a transdermal patch, wherein the transdermal patch is selected from the group such as to reservoir patch, a microreservoir patch, a matrix patch, a drug in adhesive patch, a pressure sensitive adhesive patch, extended release transdermal film, a liquid reservoir system, a microreservoir patch, a mucoadhesive patch, and combinations thereof. The disclosure provides a pharmaceutical composition indicated for the treatment and/or prevention and/or control of multiple sclerosis, multiple sclerosis-related muscle spasms, and pain and/or spasticity in multiple sclerosis in a patient. The disclosure provides a pharmaceutical composition which is formulated as the transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, once in 15 days to about 30 days. The disclosure provides a pharmaceutical composition which is formulated as microneedles. The disclosure provides a pharmaceutical composition wherein said active agent or derivative thereof is produced by a synthetic route. The disclosure provides a pharmaceutical composition co-administered with at least one additional an active agent selected from the group consisting of medications administered for treatment and/or management and/or prevention and/or control of multiple sclerosis and/or symptoms associated with multiple sclerosis. The disclosure provides a pharmaceutical composition may further comprising at least one additional active agent selected from the group consisting of: fingolimod or its salt; and teriflunomide.

The disclosure provides a transdermal and/or topical pharmaceutical composition comprising: at least one active agent selected from the group consisting of: about 0.1% to about 30% of an active agent selected from the group consisting of cannabidiol (CBD), free base forms thereof, salts thereof, isomers thereof, amorphous forms thereof, derivatives thereof, and combinations thereof; and about 0.1% to about 30% of an active agent selected from the group consisting of tetrahydrocannabinol (THC), free base forms thereof, salts thereof, isomers thereof, amorphous forms thereof, derivatives thereof, and combinations thereof, further wherein the pharmaceutical composition comprises: about 10% to about 50% of at least one solvent; about 10% to about 50% of at least surfactant; optionally, about 2% to about 30% of at least one permeation enhancer; and/or optionally, about 5% to about 80% of an adhesive and/or polymer. The disclosure provides a transdermal and/or topical pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a transdermal and/or topical pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a transdermal and/or topical pharmaceutical composition comprising one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for transdermal delivery. The disclosure provides a transdermal and/or topical pharmaceutical composition comprising one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for topical delivery. The disclosure provides a transdermal and/or topical pharmaceutical composition wherein said CBD, THC, the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a natural route or a synthetic route. The disclosure provides a transdermal and/or topical pharmaceutical composition wherein said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route. The disclosure provides a transdermal and/or topical pharmaceutical composition formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, or transdermal drug-in-adhesive matrix formulation. The disclosure provides a transdermal and/or topical pharmaceutical composition formulated as a topical liquid formulation, topical semisolid formulation, topical gel formulation, topical polymer matrix formulation, topical adhesive matrix formulation, topical film forming gel formulation, or topical film forming spray formulation. The disclosure provides a transdermal and/or topical pharmaceutical composition which is formulated as a transdermal patch. The disclosure provides a transdermal and/or topical pharmaceutical composition formulated as a transdermal patch, wherein the transdermal patch is selected from the group such as to reservoir patch, a microreservoir patch, a micro-dosing patch, a matrix patch, a drug in adhesive patch, a pressure sensitive adhesive patch, extended-release transdermal film a liquid reservoir system, a microreservoir patch, a mucoadhesive patch, and combinations thereof. The disclosure provides a transdermal and/or topical pharmaceutical composition which is formulated as a topical patch. The disclosure provides a transdermal and/or topical pharmaceutical composition formulated as a topical patch, wherein the topical patch is selected from the group such as to reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, and combinations thereof. The disclosure provides a transdermal and/or topical pharmaceutical composition which is formulated as metered dose transdermal gel, metered dose transdermal spray, a film forming gel, a film forming spray, or a meter-dose aerosol. The disclosure provides a transdermal and/or topical pharmaceutical composition formulated as microneedles. The disclosure provides a transdermal and/or topical pharmaceutical composition formulated as a liquid formulation, transdermal semisolid formulation, or transdermal polymer matrix formulation, transdermal adhesive matrix formulation, film forming gel formulation, film forming spray formulation. The disclosure provides a transdermal and/or topical pharmaceutical composition further comprising at least one additional active agent selected from the group consisting of THC, CBD, fingolimod or its salt; and teriflunomide, sertraline, paroxetine, fluoxetine, duloxetine, citalopram, docusate, mineral oil, magnesium hydroxide, bisacodyl, diazepam, tizanidine, baclofen, clonazepam, dantrolene, oxybutynin, tolterodine, tamsulosin, darifenacin, imipramine, mirabegron, solifenacine succinate, desmopressin, dalfampridine, gabapentin, carbamazepine, nortriptyline, pregabalin, oxcarbazepine, isoniazid, clonazepam, methylphenidate, modafinil, dextroamphetamine and amphetamine, modafinil, meclizine, hydroxyzine, tadalafil, sildenafil, vardenafil, ciprofloxacin, methenamine, levofloxacin, sulfamethoxazole, and combinations thereof. The disclosure provides a transdermal and/or topical pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a transdermal and/or topical pharmaceutical composition wherein the adhesive is selected from the group consisting of pressure sensitive adhesives, silicone polymers, bio psa 4302, bio-psa 4202, acrylic pressure sensitive adhesives, duro-tak 87-2156, duro-tak 387-2287, duro-tak 87-9301, duro-tak 387-2051, polyisobutylene, polyisobutylene low molecular weight, polyisobutylene medium molecular weight, polyisobutylene 35000 mw, acrylic copolymers, rubber based adhesives, hot melt adhesives, styrene-butadiene copolymers, bentonite, all water and/or organic solvent swellable polymers and combinations thereof. The disclosure provides a transdermal and/or topical pharmaceutical composition wherein said polymer is present and is selected from the group consisting of natural polymers, polysaccharides. agar, alginic acid and derivatives, cassia tora, collagen, gelatin, gellum gum, guar gum, pectin, potassium cargeenan, sodium carageenan, tragacanth, xantham, gum copal, chitosan, resin, semisynthetic polymers, cellulose, methylcellulose, ethyl cellulose, carboxymethyl cellulose, hydroxylpropyl cellulose, hydroxylpropylmethyl cellulose, synthetic polymers, carboxyvinyl polymers, carbomers, carbopol 940, carbopol 934, carbopol 971p NF, polyethylene, clays, silicates, bentonite, silicon dioxide, polyvinyl alcohol, acrylic polymers (eudragit), acrylic acid esters, polyacrylate copolymers, polyacrylamide, polyvinyl pyrrolidone homopolymer, polyvinyl pyrrolidone copolymers, PVP, Kollidon 30, poloxamer, isobutylene, ethyl vinyl acetate copolymers, natural rubber, synthetic rubber, and combinations thereof. The disclosure provides a transdermal and/or topical pharmaceutical composition wherein said permeation enhancer is present, and is selected from the group consisting of dimethylsulfoxide, dimethylacetamide, dimethylformamide, decymethylsulfoxide, dimethylisosorbide, azone, pyrrolidones, N-methyl-2-pyrrolidone, 2-pyrrolidon, esters, fatty acid esters, propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate, fatty acids, capric acid, caprylic acid, lauric acid, oleic acid, myristic acid, linoleic acid, stearic acid, palmitic acid, alcohols, fatty alcohols, glycols, oleyl alcohol, nathanol, dodecanol, propylene glycol, glycerol, ethers, alcohol, diethylene glycol monoethyl ether, urea, triglycerides, triacetin, polyoxyethylene fatty alcohol ethers, polyoxyethylene fatty acid esters, esters of fatty alcohols, essential oils, surfactant type enhancers, brij, sodium lauryl sulfate, tween, polysorbate, terpene, terpenoids, and combinations thereof. The disclosure provides a transdermal and/or topical pharmaceutical composition wherein said solvent is present, and is selected from the group consisting of methanol, ethanol, isopropyl alcohol, butanol, propanol, polyhydric alcohols, glycols, propylene glycol, polyethylene glycol, dipropylene glycol, hexylene glycol, butyene glycol, glycerine, derivative of glycols, pyrrolidone, N methyl 2-pyrrolidone, 2 pyrrolidone, sulfoxides, dimethyl sulfoxide, decymethylsulfoxide, dimethylisosorbide, mineral oils, vegetable oils, sesame oil water, polar solvents, semi polar solvents, non polar solvents, volatile chemicals, ethanol, propanol, ethyl acetate, acetone, methanol, dichloromethane, chloroform, toluene, IPA, hexane, acids, acetic acid, lactic acid, levulinic acid, bases, pentane, dimethylformamide, butane, lipids, and combinations thereof. The disclosure provides a transdermal and/or topical pharmaceutical composition which is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a transdermal and/or topical pharmaceutical composition which is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

The disclosure provides a method for the treatment and/or prevention and/or control of multiple sclerosis, multiple sclerosis-related muscle spasms, and pain and/or spasticity in multiple sclerosis in a patient comprising: selecting a patient in need of treatment and/or prevention and/or control of multiple sclerosis, multiple sclerosis-related muscle spasms, pain and/or spasticity in multiple sclerosis; topically applying the transdermal pharmaceutical composition of the disclosure. The disclosure provides a method wherein the topical application of a transdermal pharmaceutical composition for the treatment and/or prevention and/or control of multiple sclerosis, multiple sclerosis-related muscle spasms, pain and/or spasticity in multiple sclerosis in a patient, wherein the transdermal patch is applied at a time period selected from the group consisting of once in a day, once in two days, once in three days, once in four days, once in five days, once in six days, once in a week, and once in ten days. The disclosure provides a method further providing a constant rate of delivery of the active components of the transdermal patch over a time period. The disclosure provides a method further providing a steady absorption rates of the active components of the transdermal patch over a time period. The disclosure provides a method further achieving a constant blood serum levels of the active components of the transdermal patch over a time period. The disclosure provides a method further achieving a reduced variability in dosage of the active components of the transdermal patches over a time period. The disclosure provides a method further providing a plasma concentration of the active components of the transdermal patch in a therapeutic range over a period of time. The disclosure provides a method further providing a plasma concentration of the active components of the transdermal patch in a therapeutic range of about 0.5 ng/ml to about 300 ng/mL. The disclosure provides a method further providing a plasma concentration of the active components of the transdermal patch in a therapeutic range of about 0.1 ng/ml to about 100 ng/mL.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention belongs. As used herein the following terms have the following meanings.

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. Disclosure provides for, for example, transdermal formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton.
Chemical structure is shown below as formula I

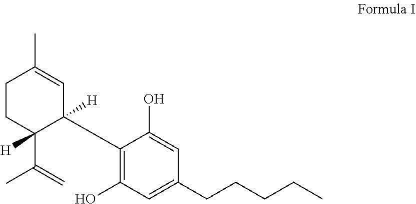 [SMILES: C=C(C)[C@@H]1CCC(C)=C[C@H]1c1c(O)cc(CCCCC)cc1O]

Tetrahydrocannbinol (THC) IUPAC Name (—)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.
Chemical structure is shown below as formula II

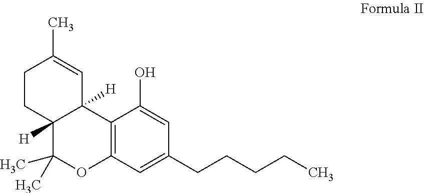 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, polymorph thereof, stereoisomers thereof, solution of THC in solvents such as but not limited to methanol, heptane, etc. alone or in combinations thereof.

As used herein, synthetic cannabinoids include at least the following:
AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl) indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl) indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54×selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl) indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl) indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor α (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28×more strongly to CB2 than CB1, and has been shown to have immunomodulatory effects, and may be useful in the treatment of pain and inflammation; JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of 48-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173—fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a, 7, 10, 10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl) naphthalen-1-ylmethane, 22 nM at CB1, JWH-176—1-([(1E)-3-pentylinden-1-ylidine]methyl) naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl) methane; JWH-185—1-pentyl-1H-indol-3-yl-(4-methoxy-1-naphthyl) methane; JWH-192—(1-(2-morpholin-4-ylethyl) indol-3-yl)-4-methylnaphthalen-1-ylmethane; JWH-193—(1-(2-morpholin-4-ylethyl) indol-3-yl)-4-methylnaphthalen-1-ylmethanone; JWH-194—2-methyl-1-pentyl-1H-indol-3-yl-(4-methyl-1-naphthyl) methane; JWH-195—(1-(2-morpholin-4-ylethyl) indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl) methane; JWH-198—(1-(2-morpholin-4-ylethyl) indol-3-yl)-4-methoxynaphthalen-1-ylmethanone; JWH-199—(1-(2-morpholin-4-ylethyl) indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic from the naphthoylindole family, which acts as a potent cannabinoid agonist at both the CB1 and CB2 receptors; JWH-213—a potent and fairly selective CB2 agonist; JWH-229—1-methoxy-3-(1′, 1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl) indole); JWH-258—a potent and mildly selective CB1 agonist; JWH-302—(1-pentyl-3-(3-methoxyphenylacetyl) indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl) indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E. coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal compositions as disclosed herein comprising, for example, biosynthetic CBD, alone or in combination with other active agents.

According to certain embodiments, transdermal compositions described herein are for the prevention and/or treatment of pain and/or inflammation. According to certain embodiments, transdermal compositions described herein are for the reduction in severity of pain and/or inflammation.

According to certain embodiments described herein, pharmaceutical composition or transdermal formulation of contains cannabidiol and/or THC—the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, solution thereof in solvents alone or in combinations thereof. More preferably transdermal formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solution of cannabidiol in methanol alone or in combinations thereof. More preferably transdermal formulation may include THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solution of cannabidiol in methanol alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites polymorphs thereof, stereoisomers thereof, coated form thereof. For example, the active agent's free base or its salts or its isomers or its amorphous form or its crystalline form or its co crystalline form or its solid solution or its prodrugs or its analogs or its derivatives or synthetic forms, polymorphs thereof, stereoisomers thereof, coated form thereof. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this invention are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term “active agent” includes the free base thereof, salts thereof, isomers thereof, amorphous forms thereof, crystalline forms thereof, co crystalline forms thereof, prodrugs thereof, analogs thereof, derivatives thereof, synthetic forms thereof, polymorphs thereof, stereoisomers thereof, coated form thereof alone or in combinations thereof. In certain embodiments the active agent is highly purified. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation in certain embodiments, the dose of active agent is greater than or equal to, for example, about 1, 2, 3, 4, 5, 6, 7, 8, 9, 10, 15, 20, 25, 30, 35, 40, or 45 mg/kg/day. In certain embodiments, the dose of active agent is equal to or greater than, for example, about 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 85%, about 90%, about 95%, about 96%, about 97%, about 98%, and about 99% of the formulation. In exemplary embodiments, formulations of the disclosure may comprise active agent at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the active agent will represent approximately 1 wt % to 75 wt %, preferably 2 wt % to 30 wt %, more preferably 5 wt. % to 20 wt % of the formulation.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

As used herein, the term “transdermal delivery” means delivery of drug into systemic circulation through the skin.

### Additional Active Agents

As used herein the term “combination administration” of a compound, therapeutic agent or known drug with the combination of the present invention means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid supressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant;

immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

Examples of active ingredients comprises drugs which are administered in multiple sclerosis to treat relapse and/or modify disease course, but is not limited to any of the following, for example, alone or in combination: teriflunomide, fingolimod, cladribine, siponimod fumaric acid, monomethyl fumarate, dimethyl fumarate, glatiramer acetate, prednisone, methylprednisolone, natalizumab, mitozantrone, etc. Examples of active ingredients for management of multiple sclerosis symptoms such as but not limited to depression (without any limitation to sertraline, paroxetine, fluoxetine, duloxetine, citalopram, etc.), bowel dysfunction (without any limitation to docusate, mineral oil, magnesium hydroxide, bisacodyl, etc.), spasticity (without any limitation to diazepam, tizanidine, baclofen, clonazepam, dantrolene, etc.), bladder dysfunction (without any limitation to oxybutynin, tolterodine, tamsulosin, darifenacin, imipramine, mirabegron, solifenacine succinate, desmopressin, etc.), gait difficulties (without any limitation to dalfampridine, etc.), pain (without any limitation to gabapentin, carbamazepine, nortriptyline, pregabalin, oxcarbazepine, etc.), tremors (such as but not limited to isoniazid, clonazepam, etc.), fatigue (such as but not limited to methylphenidate, modafinil, dextroamphetamine and amphetamine, modafinil, etc.), dizziness and vertigo (such as but not limited to meclizine, etc.), itching (such as but not limited to hydroxyzine, etc.), sexual problems (such as but not limited to tadalafil, sildenafil, vardenafil, etc.), infection (such as but not limited to ciprofloxacin, methenamine, levofloxacin, sulfamethoxazole, etc.).

More preferably transdermal delivery system comprising drug combination of two or more drugs such as but not limited to CBD, THC, fingolimod, teriflunomide, etc. Examples of drug combination for transdermal delivery system includes such as but not limited to combination of THC and CBD, combination of THC, CBD and fingolimod or its salt, combination of THC, CBD and teriflunomide, etc.

As indicated the pharmaceutical formulations as disclosed herein may comprise auxiliary excipients such as for example diluents, binders, lubricants, surfactants, disintegrants, plasticizers, tackifiers, opacifying agents, pigments, and such like. As will be appreciated by those skilled in the art, the exact choice of excipient and their relative amounts will depend to some extent on the final dosage form.

### Pharmaceutical Compositions

According to certain embodiments described herein, pharmaceutical composition or transdermal formulation of contains active agents such as THC and/or CBD, and derivatives of these compounds.

One embodiment of the present disclosure can be a transdermal drug delivery system which may include without any limitation to transdermal formulation, transdermal patches, topical formulation, microneedles, iontophoresis, metered dose transdermal spray, metered dose transdermal gel, transdermal aerosols.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch, metered dose transdermal system, sachet, etc. Transdermal formulations which includes matrix patches without any limitations like adhesive matrix patch, drug in adhesive matrix patch, non-adhesive matrix patch, a transdermal matrix formulation as drug in adhesive matrix patch is preferred.

Without any limitation, transdermal patch may include all transdermal drug delivery systems stated in art preferably but not limited to reservoir patch, matrix patch, bilayer matrix patch, multilayer matrix patch, microreservoir patch, adhesive systems, transdermally applicable tape and other.

In certain embodiments of the present disclosure, a transdermal patch comprises transdermal formulation containing active agents such as THC and/or CBD, and derivatives of these compounds contained in a reservoir or a matrix, and an adhesive which allows the transdermal patch to adhere to the skin, allowing the passage of the active agents from the transdermal patch through the skin of the patient. The transdermal delivery system can be occlusive, semi-occlusive or non-occlusive, and can be adhesive or non-adhesive.

The transdermal formulation comprising active agents can be incorporated within the patch and patch can be applied topically to the skin surface. The patch can be left on the subject for any suitable period of time.

In some embodiments, the transdermal patches provide for a constant rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a constant blood serum level of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In exemplary embodiments as disclosed herein, the transdermal patch provides a blood serum level of active agent selected from without any limitation, for example, about 0.01 ng/ml, about 0.02 ng/mL, about 0.05 ng/ml, about 0.1 ng/ml, about 0.2 ng/ml, about 0.5 ng/mL, about 1 ng/mL, about 2 ng/ml, about 5 ng/mL, about 10 ng/ml, about 20 ng/ml, about 50 ng/mL, about 100 ng/ml, about 200 ng/mL, about 500 ng/ml, about 1 μg/mL and ranges thereof. In one aspect, transdermal patch provides a blood serum level of active agent in the range of 0.01 ng/mL-400 ng/mL. In another aspect, transdermal patch provides a blood serum level of active agent in the range of 0.01 ng/ml-100 ng/mL.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation can be topically applied to the skin surface for transdermal delivery of active agents such as THC and/or CBD, and derivatives of these compounds.

The transdermal formulation and/or topical formulation of some embodiments of the present disclosure may include carriers or ingredients in effective amount either alone or in combinations thereof without any limitation to the following carriers or ingredients such as solvents, gelling agents, polymers, pressure sensitive adhesive polymers, adhesive polymers, biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, excipients, material to prepare patch, material to prepare matrix patch, material to prepare reservoir patch etc.

Active agents may be dissolved, suspended, dispersed or uniformly mixed in the above stated single carrier, mixture of carriers and combinations of carrier. Any combination of two or more drugs may be dissolved, suspended, dispersed or uniformly mixed in the above stated single carrier, mixture of carriers and combinations of carrier.

The desired optimum transdermal and/or topical formulation as disclosed herein may comprise without any limitation to following carriers as stated from example 1 to example 12 either alone or in combinations thereof.

### Indications

According to certain embodiments, transdermal compositions described herein are for the treatment and/or prevention and/or control of multiple sclerosis, multiple sclerosis-related muscle spasms, and pain and/or spasticity in multiple sclerosis.

Certain unwanted side effects caused by current medications are reduced or eliminated when treating patients with the compounds disclosed herein. One major side effect of the currently used medications containing THC (or its analogs), such as a US-listed drug dronabinol (Marinol) (synthetic THC) or Sativex, is the debilitating psychotropic effect at higher therapeutic doses. The higher doses are often necessary for resistant patients to manage pain, depression, or motoric disfunctions associated with: neuropathic pain, such as allodynia in Complex Regional Pain Syndrome (CRPS), MS spasticity, Spinal Cord Injury (SCI)-related spasticity, Hereditary Spastic Paraplegia (HSP)-related spasticity, Spastic Diplegia Cerebral Palsy (SDCP)-related spasticity, Spinocerebellar Ataxias (SA)-related spasticity, Huntington's Disease (HD) chorea, Post Traumatic Stress Disorder (PTSD) depression, Tuberous Sclerosis (TS) tumors and inflammation, and others.

Neurodegenerative diseases, such as MS, are characterized by changes in normal neuronal functioning, leading, in most cases, to neuronal death. Studies in animals and humans demonstrate that changes also occur in the endocannabinoid. More specifically, multiple studies indicate that control of spasticity in MS is mediated by CB1, and not CB2 cannabinoid receptors, but the role of CB2 receptor cannot be entirely excluded.

There are multiple studies demonstrating spasticity inhibition of THC—the main psychotropic constituent of the Cannabis sativa plant (Petro & Ellenberger, Treatment of human spasticity with delta 9-tetrahydrocannabinol, 1981) (Ungerleider, Andyrsiak, Fairbanks, Ellison, & Myers, Delta-9-THC in the treatment of spasticity associated with multiple sclerosis, 1987). Also, a synthetic THC drug Marinol (United Pharmaceuticals) and THC derived Cesamet (Valeant Pharmaceuticals International) were tested and both demonstrated positive results in reducing spasticity. This is not surprising since THC is a CB1 and CB2 receptor partial agonist. Yet, CB1 receptor also mediates the THC-induced psychoactivity. Consequently, there is a mounting evidence of the unwanted psychotropic effects, especially with synthetic THC and natural THC at higher doses, such as intoxication, sedation, memory impairment, dysphoria (and adverse behavioral changes.

It is rather difficult to dissociate the therapeutic effects from the adverse effects of cannabinoids when using cannabis as a medicine. It is, however, hypothesized that this aspect can be mitigated by administering a cannabis extract that contains other cannabinoids in addition to THC. Cannabidiol—an antagonist of CB1 and CB2 receptors is believed to be modulating the THC psychotropic effect. However, the effective concentrations and ratios, pharmacodynamics, presence or absence of other substances that facilitate this complex interaction was not understood until now.

It is plausible that CBD functions as a negative allosteric modulator by binding to a site on the CB1 receptor distinct from the THC binding site. From this secondary site, the inventors conclude, CBD is able to change the shape of the receptor in a way that there is less THC binding and less activation of the CB1 receptor. CBD was shown to inhibit uptake and metabolism of anandamide. It is also suggested that at low concentrations CBD acts as an inverse agonist, binding to the same receptors that other agonists do but causing a different physiological effect. Reduced THC potency was seen at CBD concentrations as low as 100 nM that is well achievable in a human. Further, in line with classical pharmacology, the responses THC elicits appear to be strongly influenced both by the expression level and signaling efficiency of cannabinoid receptors and by ongoing endogenous cannabinoid release.

CBD also inhibits the conversion of THC into its particularly psychoactive metabolites 11-hydroxy-THC, reducing psychoactive effects, yet, it works synergistically with THC on control of pain and spasticity. It is further suspected that CBD delays the absorption of THC, and consequently peak serum concentrations that are associated with the occurrence of unwanted side effects are avoided. It has been further observed that cannabis strains, containing CBD levels equal or higher than THC, have positive effects on muscle spasticity. These positive effects of cannabis on spasticity and pain and its safety have also been emphasized by the American Academy of Neurology (Koppel, et al., 2014). Moreover, CBD is presumed to have antipsychotic, anxiolytic, and anticonvulsant effects, and experimental evidence suggests that THC, as well as CBD, exhibit anti-inflammatory, neuroprotective, and immunomodulatory properties, pointing to additive or potentiating effects of the combination of the two.

Considering the known deficiencies of comparable commercial and experimental drugs, disclosed herein are new formulations that are more effective clinically for treating pain, inflammation, mood, spasticity, and motoric symptoms associated with psychiatric, autoimmune and neurological diseases and disorders, such as, but not limited to neuropathic, acute or chronic pain such as in CPRS, cancer or trauma; Ataxias (cerebellar, sensory, vestibular), such as tremor or vertigo; MS, HSP, SCI, SDCP, SA spasticity and pain, HD chorea; PD rigidity; PTSD depression; migraine; TS tumors and inflammation—all these symptoms are responsive to specific modulations of the endocannabinoid receptors and/or the neuroprotective/antioxidant qualities of specific cannabinoids.

Though, the clinical effect of combination therapy has been confirmed in studies and commercially with Sativex (THC/CBD combination where THC>CBD), it is apparent that the total dose of THC delivered in Sativex is not sufficient to saturate the receptors, not making full use of the medication's therapeutic potential. The literature on Sativex shows a large variance in daily doses. Sativex is administered as buccal spray, from 5 sprays per day (Johnson, Lossignol, Burnell-Nugent, & Fallon, 2013) to a maximum of 14 sprays (THC 2.7 mg: CBD 2.5 mg per spray) (Bayer HealthCare press release and Medical Update Memo of Multiple Sclerosis Society of Canada, 2005). At these high doses, not surprisingly, the unwanted psychotropic effects of THC become dominant, limiting the drug's utility.

To achieve the desired efficacy attainable at high THC doses, but overcoming the negative psychotropic effect, the inventor employs a balanced THC/CBD formula that ranges in some embodiments 0.5 to 1 (THC) and 1.5 to 2 (CBD) ratios, and in other embodiments 1 to 1.8 (THC) and 2 to 2.5 (CBD) ratios (from approximately 52% to 83% of CBD, where THC and CBD is 100%), whereas CBD in the ratios significantly higher than found in Sativex (relative to THC concentrations), would limit the THC psychotropic effect. In addition, in one embodiment, the formulation contains up to 50% of the total formulation other phytochemicals and impurities co-extracted with THC and/or CBD from the Cannabis plant. And in another embodiment, THC and CBD represent at least 97% of the total formulation and the remaining 3% or less are impurities.

Considering the safety aspects associated with higher THC and CBD doses, the inventor believes, and it was experimentally determined in humans and animals, that aforesaid formulations and dosing is appropriate for patients, which is in addition corroborated by the following experiments with higher THC ratios and, in one case, a 200 mg high CBD dose: 200 mg CBD per day or 10 mg THCV per day or 10 mg CBD and 10 mg THCV per day or 200 mg CBD and 10 mg THCV per day were all well tolerated.

In one embodiment of the proposed invention, the treatment of a patient with an autoimmune disease Rheumatoid arthritis involves giving to a patient every 12 hours by oral administration one ARCO® Chemie 60 Oval soft-gel capsule of the compound containing a mixture of 5 mg delta-9 and delta-8-tetrahydrocannabinol (THC) and cannabidiol (CBD) in the ratio of approximately 0.7 (THC) and 2 (CBD) by mass, and such mixture contains less than 5 mg of other cannabinoids co-extracted with THC and CBD from the Cannabis indica plant and not completely removed from the extract, and in one embodiment, one or more non-cannabinoid components, such as sesame oil and a mixture of parahydroxybenzoates, where such capsule is a time-released capsule designed to release said compound in the small intestine; and in another embodiment, in the stomach. The aforesaid compound and method have statistically meaningfully reduced perception of pain on the Rheumatoid Arthritis Pain Scale (RAPS) in subjects suffering from pain associated with Rheumatoid Arthritis inflammation after being administered for 5 days, having minimal to nonexistent debilitating psychotropic effect. RAPS measures physiological, sensory-discriminative, and cognitive components.

The invention will be illustrated in more detail with reference to the following Examples, but it should be understood that the present invention is not deemed to be limited thereto.

### EXAMPLES

### Example 1

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, sesame oil, water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane, etc.), pentane, dimethylformamide, butane, lipids, acids such as but not limited to acetic acid, lactic acid, levulinic acid, bases and others. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 95% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

### Example 2

The transdermal formulation and/or topical formulation of the disclosure may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.), acrylic pressure sensitive adhesives such as but not limited to (duro-tak 87-2156, duro-tak 387-2287, duro-tak 87-9301, duro-tak 387-2051 etc.), polyisobutylene such as but not limited to (polyisobutylene low molecular weight, plyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymers and/or pressure sensitive adhesive polymers will represent approximately 1 wt % to 75 wt %, preferably 2 wt % to 30 wt %, more preferably 5 wt. % to 20 wt. % of the formulation, and more preferably in the range of 0.1% 80% w/w or w/v.

### Example 3

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate, lauryl lactate, ethyl oleate, etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 4

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate (e.g., TWEEN®) such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxy 1-6-glycerides, lauroyl polyoxyl-6-gylcerides, polyglycery 1-3-dioleate, diethylene glycol monoethyl ether, propylene glycol monolaurate type I, polyglyceryl-3-dioleate, caprylocaproyl polyoxyl-8 glycerides etc, cyclodextrins and others. More preferably in the range of 0.01% 95% w/w or w/v. In exemplary embodiments, formulations of the disclosure may comprise solubilizers, surfactants, emulsifying agents, dispersing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, and more preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, packaging of transdermal delivery system, moisture scavengers, etc. Different stabilizers can be used to increase stability of active agents in formulation.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation, and more preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, and more preferably in the range of 0.01%-50% w/w or w/v.

### Example 10

The transdermal formulation and/or topical formulation of the disclosure may be formulated in forms such as in ointment base, cream base, gel base, known to those skilled in the art.

### Example 11

Materials to make the transdermal delivery system of the disclosure in patch form known to those skilled in the art, for example, such as but not limited to reservoir patch, matrix patch, drug in adhesive matrix patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of active agent, such as for example, THC and/or CBD, and derivatives of these compounds, alone or in combinations thereof in human plasma required for treating and/or preventing pain and/or inflammation. Therapeutically effective active agent such as THC and/or CBD, and derivatives of these compounds dosages refers to the therapeutic concentration of THC and/or CBD in human plasma required for treating and/or preventing and/or controlling of multiple sclerosis, multiple sclerosis-related muscle spasms, and pain and/or spasticity in multiple sclerosis in a patient. Furthermore, the precise therapeutic effective dose of such as CBD and/or THC, and derivatives of these compounds in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

The transdermal formulation or transdermal patch of active agents such as CBD and/or THC, and derivatives of these compounds can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

The transdermal formulation and/or topical formulation of the invention may comprise fillers such as but not limited to colloidal silicon dioxide, lactose, mannitol, talc, titanium dioxide, etc. clays such as but not limited to kaolin, bentonite, etc, etc. either alone or in combinations thereof. More preferably in the range of 0.01%-70% w/w or w/v.

### Example 13

The transdermal formulation and/or topical formulation of the invention may comprise crystallization inhibitors, tackifiers, cross-linking agents, resins etc. either alone or in combinations thereof.
The transdermal formulation and/or topical formulation and/or transdermal delivery system of the disclosure may deliver at least therapeutic effective dose of cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof. Therapeutic effective cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof in human plasma required for treating and/or preventing pain and/or inflammation. Therapeutic effective cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof dose refers to the therapeutic concentration of these forms of cannabidiol in human plasma required for treating and/or preventing pain and/or inflammation. Furthermore, the precise therapeutic effective dose of cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof in the transdermal formulation or topical formulation or transdermal delivery system or transdermal patch can be determined by those skilled in the art based on factors such as but not limited to the patient's condition etc. The transdermal formulation or topical formulation or transdermal delivery system or transdermal patch will be available in different dosage strengths and patch sizes in order to achieve optimum therapeutic outcome based on patient's requirement.
In yet another embodiment, the transdermal formulation and/or topical formulation and/or transdermal delivery system or transdermal patch of the disclosure may deliver at least therapeutic effective dose of cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof. Therapeutic effective cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof refers to the therapeutic concentration of cannabidiol, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, alone or in combinations thereof in human plasma required for treating and/or preventing and/or controlling the conditions and/or diseases and/or disorders associated with multiple sclerosis, multiple sclerosis-related muscle spasms, pain and/or spasticity in multiple sclerosis.

### Example 14

Examples of transdermal formulations of drug in adhesive matrix patch such as but not limited to (Example Transdermal Formulations of drug in adhesive matrix patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |
| Polymer | 1%-10% |

### Example Transdermal Formulations of Drug in Adhesive Matrix Patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

### Example 15

Synthetic cannabidiol (CBD) formulations for transdermal delivery ((Formulation Nos. 001, 002, 003, 004, and 005) were prepared by mixing ingredients as shown in Table 1:

TABLE 1
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 9.35 | 9.06 | 9.34 | 9.09 | 9.13 |
| PG | 90.65 | 45.51 | 45.17 | 45.54 | 45.33 |
| Hexylene | | 45.43 | | | |
| Glycol | | | | | |
| 1,3 | | | 45.49 | | |
| Butanediol | | | | | |
| PEG-400 | | | | 45.37 | |
| Dipropylene | | | | | 45.53 |
| Glycol | | | | | |

Abbreviations: PG = propylene glycol; CBD = Cannabidiol; PEG-400: Polyethylene Glycol-400.

All of the components from Table 1, with the exception of the CBD, were mixed together with stirring for 18 hours. Next, the CBD was added into the excipient mixture to prepare the final transdermal formulations.
The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 5:

TABLE 2
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24, 48, 72, 96, 120, 144 |
| Franz-cell diffusion area (sqcm) | 1.76 |
| Membrane Type | Human Cadaver Skin |

Flux of CBD through the human cadaver skin was measured for a minimum period of 144 Hrs (6 days) and results of the flux measurement are provided in Table 6.

TABLE 3
CBD Flux Results

| | 001 | 002 | 003 | 004 | 005 |
|---|---|---|---|---|---|
| Total Amount of CBD | 85795 | 167045 | 150000 | 59091 | 166477 |
| Permeated at 144 hrs | | | | | |
| (ng/cm²) | | | | | |
| Flux (ng/cm²/hr) | 338.5 | 1160.03 | 1041.66 | 410.35 | 1156.09 |

### Example 16

Additional synthetic Cannabidiol (CBD) formulations for transdermal delivery (Formulation Nos. 006 through 014) were prepared by mixing ingredients as shown in Table 4:

TABLE 4
Transdermal Synthetic Cannabidiol formulation no. 015 to 022

| | 015 | 016 | 017 | 018 | 019 | 020 | 021 | 022 |
|---|---|---|---|---|---|---|---|---|
| Ingredients | (% W/W) | (% W/W) | (% W/W) | (% W/W) | (% W/W) | (% W/W) | (% W/W) | (% W/W) |
| CBD | 9.95 | 9.64 | 9.77 | 9.98 | 9.98 | 9.64 | 9.87 | 9.52 |
| PG | 42.70 | 42.58 | 42.51 | 42.51 | 42.02 | 42.45 | 42.47 | 42.34 |
| 1,3 Butanediol | 42.36 | 42.48 | 42.40 | 42.63 | 42.66 | 42.77 | 42.50 | 42.64 |
| Tween-20 | 4.99 | | | | | | | |
| Triacetin | | 5.30 | | | | | | |
| PGML | | | 5.32 | | | | | |
| OA | | | | 4.88 | | | | |
| ML | | | | | 5.34 | | | |
| IPM | | | | | | 5.16 | | |
| IPP | | | | | | | 5.04 | |
| Labrasol | | | | | | | | 4.91 |

Abbreviations: CBD = Cannabidiol; PGML: Propylene glycol monolaurate; PG = propylene glycol; OA = Oleyl Alcohol; ML = Methyl Laurate; IPM = Isopropyl Myristate; IPP: Isopropyl Palmitate.

Synthetic Cannabidiol formulations for transdermal delivery (006-014) were prepared by the same procedure described in Example 1. Flux measurement was also performed as described in Example 1. The experimental conditions are the same as provided in Table 2 of Example 1.
Flux of CBD through the human cadaver skin was measured for a minimum period of 48 Hrs and results of the flux measurement experiments are provided in Table 5.

TABLE 5
CBD Flux Results

| | Formulation No. | | | | | | | |
|---|---|---|---|---|---|---|---|---|
| | 015 | 016 | 017 | 018 | 019 | 020 | 021 | 022 |
| Total Amount of | 25791 | 22851 | 29098 | 37085 | 37351 | 45008 | 59954 | 21524 |
| CBD Permeated at | | | | | | | | |
| 48 hrs (ng/cm²) | | | | | | | | |
| Flux (ng/cm²/hr) | 537.31 | 476.06 | 606.20 | 772.60 | 778.14 | 937.67 | 1249.04 | 448.41 |

### Example 17

Additional synthetic cannabidiol (CBD) formulations for transdermal delivery patches (Formulation Nos. 015 to 018) were prepared by mixing ingredients as shown in Table 6:

TABLE 6
Transdermal Synthetic cannabidiol formulation nos. 023 to 026

| | 023 | 024 | 025 | 026 |
|---|---|---|---|---|
| Ingredients | (% WAV) | (% WAV) | (% WAV) | (% WAV) |
| CBD | 2.0 | 2.0 | 2.0 | 2.0 |
| PG | 27.8 | 27.8 | 27.8 | 27.8 |
| 1,3 Butane diol | 27.8 | 27.8 | 27.8 | 27.8 |
| Durotak 9301 | 42.4 | | | |
| Durotak 2516 | | 42.4 | | |
| Durotak 2207 | | | 42.4 | |
| Silicone Adhesive | | | | 42.4 |

To prepare a transdermal patch containing synthetic cannabidiol, all of the components from Table 6, with the exception of the CBD, were mixed together with stirring for 18 hours. Next, the CBD was added 30 minutes before spreading the formulation. The formulation was spread using a commercial benchtop spreader. Specifically, the formulation matrix is evenly spread onto an 8×14 inch sheet of release liner (such as 3M 9744) to a thickness of 0.5 mm. The sheet is then place in an oven at 100° F. for one hour to evaporate off the ethyl acetate and ethanol adhesive solvent. An opaque backing membrane (such as 3M 9730 NR film) with low permeability to oxygen, for inhibition of photo and oxidative degradation, is then carefully applied to the sheet by hand to avoid formation of bubbles and voids. A circular die (1.5 inches diameter) was used to cut patches (7 cm²) for subsequent studies.

The general procedure for flux measurements of transdermal formulations in the examples above was as follows. The human cadaver skin, stored at −80° C., was thawed at room temperature in PBS, and visually inspected for defects before use. Transdermal flux was measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The general procedure for flux measurement of the transdermal adhesive patch is as follows. The release liner is peeled off the patch and the adhesive surface is applied to a piece of human cadaver skin. The transdermal patch was adhered to the skin with the patch on the side of the skin in contact with the donor compartment. The receptor compartment was filled with receptor medium, held at constant temperature, and constantly stirred to collect the CBD as it diffuses from the adhered patch, through the skin and into receptor compartment. It was confirmed that the receptor fluid was always in contact with the skin. The receptor compartment was emptied at 24 hour intervals for assay of CBD and replaced with fresh receptor solution. In order to maintain the sink condition in receptor compartment, the CBD concentration in the receptor compartment was maintained at less than 10% of its solubility. The experimental conditions are the same as provided in Table 2 of Example 15.

### Example 18

Synthetic cannabidiol (CBD) formulations for transdermal delivery ((Formulation Nos. 047-055) were prepared by mixing ingredients as shown in Table 7:

TABLE 7
Transdermal Synthetic Cannabidiol formulations

| Excipients | CBD 047 | CBD 048 | CBD 049 | CBD 050 | CBD 051 | CBD 052 | CBD 053 | CBD 054 | CBD 055 |
|---|---|---|---|---|---|---|---|---|---|
| CBD | 4.84% | 4.98% | 4.73% | 4.99% | 4.87% | 4.89% | 5.04% | 4.83% | 5.00% |
| DURO- | 95.16% | — | — | — | — | — | — | — | — |
| TAK 2516 | | | | | | | | | |
| DURO- | — | 95.02% | — | — | — | — | — | — | — |
| TAK 9301 | | | | | | | | | |
| DURO- | — | — | 95.27% | — | — | — | — | — | — |
| TAK 2287 | | | | | | | | | |
| DURO- | — | — | — | 95.01% | — | — | — | — | — |
| TAK 2054 | | | | | | | | | |
| DURO- | — | — | — | — | 95.13% | — | — | — | — |
| TAK 2852 | | | | | | | | | |
| DURO- | — | — | — | — | — | 95.11% | — | — | — |
| TAK 2074 | | | | | | | | | |
| DURO- | — | — | — | — | — | — | 94.96% | — | — |
| TAK 2194 | | | | | | | | | |
| BIO-PSA | — | — | — | — | — | — | — | 95.17% | — |
| 4501 | | | | | | | | | |
| BIO-PSA | — | — | — | — | — | — | — | — | 95.00% |
| 4201 | | | | | | | | | |

The above ingredients (Table 7) are blended by stirring for 18 hours and then, using a commercial benchtop spreader, the matrix is evenly spread onto an 8×14 inch sheet of release liner (such as 3M 9744) to a thickness of 0.5 mm. The sheet is then place in an oven at 86 F for 120 min to evaporate off the ethyl acetate adhesive solvent. An opaque backing membrane (such as 3M 9730 NR film) with low permeability to oxygen to inhibit photo and oxidative degradation, is then carefully applied by hand to avoid formation of bubbles and voids. A circular die (1.5 inches diameter) is used to cut patches (1.76 sqcm) for subsequent studies. After drying, the drug adhesive matrix has a surface density of 2-30 mg/sqcm, containing CBD in 5% w/w.
The prepared formulations where then subjected to a release study as follows: After weighing the patches (n=3), the release liner was removed, and the patches were placed in 20 ml scintillation vials with 15 ml of receiving media. The receiving media was PBS solution of pH 7.4 with 0.5% Brij (O) 20. Vials were placed on the roller overnight at 20 RPM. Samples were withdrawn every 24 hours, up to 72 hours, and media was fully replaced each time. Samples were then run in the HPLC in order to determine the % release of CBD from the different formulations.
The prepared formulations also analyze for the uniformity of drug content. The patches (n=3) were weight out for each formulation, the release liner was removed, and the patches (including the release liner) were placed in 20 ml scintillation vials with 15 ml of solution IPA:Ethanol (190proof) (50:50). The vials were then placed on the roller at 20 RPM and left overnight. Samples were withdrawn from each vial and analyzed on the HPLC in order to determine the drug content of each formulation.

TABLE 8
Experimental Condition for In-vitro Permeability testing

| | PBS (pH 7.4) + 0.5% |
|---|---|
| | Brij-O(20) + 0.01% |
| Receiving Media | Sodium Azide |
| Receiving Media Volume (mL) | 15 |
| Sample Volume (mL) | 15 |
| Sampling Interval (hr) | 24, 48 |
| Diffusion area (sqcm) | 1.76 |

% Release of CBD through the matrix system was measured for a minimum period of 48 Hrs (2 days) and results of the % release are provided in Table 9.

TABLE 9
% CBD Release Results

| | CBD 047 | CBD 048 | CBD 049 | CBD 050 | CBD 051 | CBD 052 | CBD 053 | CBD 054 | CBD 055 |
|---|---|---|---|---|---|---|---|---|---|
| Av. % Release | 26 (3) | 9 (7) | 20 (7) | 40 (2) | 23 (8) | 35 (12) | 37 (6) | 93 (2) | Did not |
| at 24 hours | | | | | | | | | solubilize |
| Av. % Release | 18 (7) | 6 (12) | 13 (13) | 22 (1) | 13 (9) | 12 (1) | 22 (5) | 2 (2) | the CBD |
| at 48 hours | | | | | | | | | 98% |
| % Release | 44 | 15 | 33 | 62 | 36 | 47 | 59 | 95 | (0.05) |
| 0-48 hours | | | | | | | | | |
| (cumulative) | | | | | | | | | |
| % CBD from | 104 (2) | 100 (1) | 100 (5) | 102 (1) | 103 (6) | 96 (15) | 104 (4) | 107 | |
| extraction | | | | | | | | | |
| (STD) | | | | | | | | | |

Drug content study showed that the % recover of CBD in extraction is between 96-107% for all the manufactured formulations. Furthermore, the release study showed that the silicone adhesive 4501 showed more than 90% release within first 24 hrs. Based on the release profile following are the best adhesive for CBD formulation: BIOPSA-4501>2054=2194>2074>2516>2852=2287>9301.

Release studies indicate that the functional group and crosslinker affect the CBD release from acrylic adhesive. According to current study, acrylic adhesive containing—COOH functional group with crosslinker showed the maximum release of CBD from all the acrylic adhesive patches.

### Example 19

Additional synthetic Cannabidiol (CBD) formulations for transdermal delivery (Formulation Nos. 057 through 064) were prepared by mixing ingredients as shown in Table 10:

TABLE 10
Transdermal Synthetic Cannabidiol formulation no. 057 to 064

| Excipients | CBD 057 | CBD 058 | CBD 059 | CBD 060 | CBD 061 | CBD 062 | CBD 063 | CBD 064 |
|---|---|---|---|---|---|---|---|---|
| CBD | 5.0% | 5.0% | 5.0% | 4.9% | 4.8% | 4.8% | 4.9% | 4.8% |
| BIO-PSA | 95.0% | 84.6% | 84.5% | 85.0% | 83.3% | 89.7% | 86.9% | 89.6% |
| 4501 | | | | | | | | |
| IPM | — | 10.4% | — | — | — | — | — | — |
| IPP | — | — | 10.5% | — | — | — | — | — |
| Oleic Acid | — | — | — | 10.1% | — | — | — | — |
| Transcutol P | — | — | — | — | 11.9% | — | — | — |
| Brij O20 | — | — | — | — | — | 5.5% | — | — |
| Poloxamer | — | — | — | — | — | — | 8.2% | — |
| 124 | | | | | | | | |
| PGML | — | — | — | — | — | — | — | 5.6% |

Synthetic Cannabidiol formulations for transdermal delivery (057-064) were prepared by the same procedure described in Example 17.
The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 11:

TABLE 11
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24, 48, 72, 96 |
| Franz-cell diffusion area (sqcm) | 1.76 |
| Membrane Type | Human Cadaver Skin |

Flux of CBD through the human cadaver skin was measured for a minimum period of 96 Hrs (4 days) and results of the flux measurement are provided in Table 12.
Upon completion of the flux study, the used patches were carefully removed and extract the CBD from the use patches using IPA:Ethanol (50:50). The human cadaver skin was also soaked in IPA:Ethanol (50:50), in order to extract the CBD from it. The samples were analyzed using HPLC. The data in Table 12 showed the amount of CBD present in the skin and the left-over patches.

TABLE 12
CBD Flux Results

| Excipients | CBD 057 | CBD 058 | CBD 059 | CBD 060 | CBD 061 | CBD 062 | CBD 063 | CBD 064 |
|---|---|---|---|---|---|---|---|---|
| Av. cumulative | 52 | 31 | 16 | 120 | 86 | BLLQ | BLLQ | 53 |
| amount permeated at | | | | | | | | |
| 96 hrs (μg) | | | | | | | | |
| Av. flux 24- | 0.41 | 0.37 | 0.37 | 0.69 | 0.51 | BLLQ | BLLQ | 0.42 |
| 96 hrs (μg/hr/sqcm) | | | | | | | | |
| Peak flux | 0.44 | 0.38 | 0.38 | 0.82 | 0.60 | BLLQ | BLLQ | 0.44 |
| (μg/hr/sqcm) | | | | | | | | |
| Time to peak flux | 72 | 72 | 72 | 72 | 72 | 0 | 0 | 72 |
| (hrs) | | | | | | | | |

| | | | | | | | |
|---|---|---|---|---|---|---|---|
| Av. CBD Amount in | 1.57 | Did not Determine | 1.14 | 2.26 | 1.81 | 1.88 | 1.56 |
| patch (mg) | | | | | | | |
| Av. CBD Amount in | 0.01 | | 0.59 | 0.13 | 0.54 | 0.24 | 0.10 |
| skin (mg) | | | | | | | |

### Example 20

The effect of gelling agents and their concentration on the permeation of CBD through human cadaver skin. CBD gel formulation can be gelled by gelling agents including but not limited to, natural polymers such as natural polymers, polysaccharides and its derivatives such as but not limited to (agar, alginic acid and derivatives, cassia tora, collagen, gelatin, gellum gum, guar gum, pectin, potassium or sodium carrageenan, tragacanth, xanthum gum, copal, starch, chitosan, resin etc.), synthetic polymers and its derivatives such as without any limitation to carboxyvinyl polymers or carbomers (carbopol 940, carbopol 934, carbopol 971), polyethylene and its copolymers etc. clays such as silicate etc. polyvinyl alcohol, polyacrylamide, polyvinyl pyrrolidone homopolymer and polyvinyl pyyrolidone copolymers (PVP, Poloxamer), acrylic acid its ester, polyacrylate copolymers, isobutylene, ethylene vinyl acetate copolymers, natural rubbers, synthetic rubbers such as styrene-diene copolymers, styrene-butadiene block copolymers, isoprene block copolymers, acrylonitrile butadiene rubber, butyl rubber or neoprene rubber, as well as pressure sensitive adhesive based on silicone, or “hot-melt adhesive”. In addition, other than human cadaver skin, CBD can be evaluated with other artificial membranes including but not limited to cellulose membrane, silicone membranes (polydimethylsiloxane), liposome coated membranes, solid-supported liquid membranes, lecithin organogel membrane and other. Besides the gel formulation of CBD, other dosage forms including but not limited to ointment, creams, emulsion, liposomes, etc. may be used.

### Example 21

The effect of enhancers or solubilizers on the flux of CBD through human cadaver skin was evaluated. The desire optimum composition of CBD gel formulation contained dimethylsulfoxide (DMSO), dimethylisosorbide (DMI), Lactic acid, Tween-20, highly purified diethylene glycol monoethyl ether (Transcutol P), dipropylene glycol, polyethylene glycol-400, propylene glycol (PG), Hexylene Glycol (HG), Lauroglycol-90. Apart from above mentioned enhancers and/or solubilizers, the CBD transdermal delivery can be influenced by enhancers and/or solubilizers including but not limited water, sulfoxides, and similar chemicals such as but not limited to (dimethylsulfoxide, dimethylacetamide, dimethylformamide, decylmethylsulfoxide, dimethylisosorbide etc), azone, pyrrolidones such as but not limited to (N-methyl-2-pyrrolidone, 2-pyrrolidon etc), esters such as but not limited to (Propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate etc), fatty acids such as but not limited to (capric acid, caprylic acid, lauric acid, oleic acid, myristic acid, linoleic acid, stearic acid, palmitic acid etc), alcohols, fatty alcohols and glycols such as but not limited to (oleyl alcohol, nathanol, dodecanol, propylene glycol, glycerol etc), ethers such as but not limited to (diethylene glycol monoethyl ether), urea, polyoxyethylene fatty alcohol ethers, polyoxyethylene fatty acid esters, esters of fatty alcohols, esters of long chain fatty acids with methyl, ethyl or isopropyl alcohol, esters of fatty alcohols with acetic acid, lactic acid, as well as oleic acid diethanolamine, essential oils, terpene and terpenoids such as but not limited to (terpineol, limonene, thymol, cineole etc), surfactant type enhancers (polysorbate 80, polysorbate 20 etc.), liposomes, niosomes, transferomes, ethanosomes, polysorbate such as but not limited to (polysorbate 20, polysorbate 40, polysorbate 60, polysorbate 80 etc), span such as but not limited to (span 80, span 20 etc), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, Caprylic glyceride, oleoyl-polyoxyl-6-glycerides, lauroyl polyoxyl-6-gylcerides, polyglyceryl-3-dioleate, diethylene glycol monoethyl ether, propylene glycol monolaurate type I etc, cyclodextrins, polyhydric alcohol, especially 1,2-propanediol, butanediol, glycerine, polyethylene glycol (m.w. 100 and higher), Dimethyl Sulfoxide, Dimethyl Isosorbide, tetrahydrofurfuryl alcohol, diethyl tolumide, monoisopropylidene glycerine and others Solubilizers, surfactants, emulsifying agents, dispersing agents and similar compounds or chemicals known to those skilled in the art can be used either alone or in combination thereof

### Example 22

Oral bioavailability of CBD is only 13-19%. For our calculation purpose, we took an average bioavailability of 15%¹⁷. So, the actual dose delivering to patient upon oral delivery is described in Table 13.

TABLE 13
Theoretical dose required from Transdermal Dosage form.

| Oral Dose | Transdermal Dose range (mg/day) |
|---|---|
| 6.2 mg/day | 0.93 |
| 62 mg/day | 9.3 |

Flux Required = Dose/Surface area
=0.93 mg/day/surface area
=930 ug /24 hr/50 sqcm
=0.78 ug/sqcm/hr

So, 50 sqcm patch with 0.78 ug/sqcm/hr flux will deliver 0.93 mg of drug in one day through transdermal route which is equivalent to 6.2 mg/day oral dose. As we know that 6.2 mg/day dose is very effective to reduce knee-joint swelling. The current formulation can deliver required amount of CBD to reduce the arthritis pain.
Some research article showed oral bioavailability of CBD is in the range of 5-6%^{17,18}, which indicates that it might reduce patch size based on first-in-human bioavailability data.

While the invention has been described in detail and with reference to specific examples thereof, it will be apparent to one skilled in the art that various changes and modifications can be made therein without departing from the spirit and scope thereof.

### REFERENCE

1) Sativex oromucosal spray SmPc www.medicines.org.uk/emc/product/602 Accessed Apr. 8, 2020
2) National Multiple Sclerosis Society “medications” www.nationalmssociety.org/Treating-MS/Medications, accessed on Apr. 8, 2020

What is claimed is:

1. A method for the treatment and/or prevention and/or control of multiple sclerosis, multiple sclerosis-related muscle spasms, and pain and/or spasticity in multiple sclerosis in a patient comprising:
selecting a patient in need of treatment and/or prevention and/or control of multiple sclerosis, multiple sclerosis-related muscle spasms, pain and/or spasticity in multiple sclerosis;
topically applying a transdermal pharmaceutical composition comprising:
an active agent consisting essentially of synthetic delta-9 tetrahydrocannabinol (THC) which has a purity of at least about 99.75% (w/w) wherein the concentration of synthetic delta-9 THC in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w,
further wherein the pharmaceutical composition comprises:
about 5% to about 5% w/w of a solvent comprising propylene glycol;
about 10% to about 50% w/w of at least one surfactant comprising polyoxyethylene fatty acid esters;
about 5% to about 15% w/w of at least one penetration enhancer comprising oleic acid;
about 50% to about 80% w/w of pressure sensitive silicone adhesive;
about 2% to about 15% w/w of at least one suspending agent comprising silicon dioxide; and
about 0.1%, 0.2%, 0.3%, 0.4%, 0.5% or 0.6% of an antioxidant comprising butylated hydroxytoluene (BHT),
wherein the pharmaceutical composition is in the form of a transdermal matrix patch and provides a constant flux of the synthetic delta-9 THC to provide a plasma concentration of the synthetic delta-9 THC in a therapeutic range in a patient over at least 7 days.

2. The method of claim 1 wherein the topical application of a transdermal pharmaceutical composition for the treatment and/or prevention and/or control of multiple sclerosis, multiple sclerosis-related muscle spasms, pain and/or spasticity in multiple sclerosis in a patient, wherein the transdermal patch is applied at a time period selected from the group consisting of once in a day, once in two days, once in three days, once in four days, once in five days, once in six days, once in a week, and once in ten days.

3. The method of claim 1 further providing a constant rate of delivery of the active components of the transdermal patch over a time period.

4. The method of claim 1 further providing a steady absorption rates of the active components of the transdermal patch over a time period.

5. The method of claim 1 further achieving a constant blood serum levels of the active components of the transdermal patch over a time period.

6. The method of claim 1 further achieving a reduced variability in dosage of the active components of the transdermal patches over a time period.

7. The method of claim 1 further providing a plasma concentration of the active components of the transdermal patch in a therapeutic range over a period of time.

8. The method of claim 1 further providing a plasma concentration of the active components of the transdermal patch in a therapeutic range of about 0.5 ng/mL to about 300 ng/mL.

9. The method of claim 1 further providing a plasma concentration of the active components of the transdermal patch in a therapeutic range of about 0.1 ng/ml to about 100 ng/mL
